(19) United States
(12) Patent Application Publication
(10) Pub. No.: US 2011/0151027 A1
(43) Pub. Date: Jun. 23, 2011

(54) STRONTIUM-DOPED CALCIUM PHOSPHATE BONE GRAFT MATERIALS

(76) Inventors: Theodore D Clineff, Phoenixville, PA (US); Justin J. Martin, Phoenixville, PA (US); Matthew B. Havener, Conshohocken, PA (US); Marissa M. Darmoc, Philadelphia, PA (US)

(21) Appl. No.: 12/643,218

(22) Filed: Dec. 21, 2009

(51) Int. Cl. A61K 33/00; A61P 19/00

(52) U.S. Cl. 424/722

(57) ABSTRACT

The present invention is directed to strontium-containing calcium phosphate bone graft materials. Methods of making and using the bone grafts are also described.

### TECHNICAL FIELD

The present invention is directed to strontium-containing calcium phosphate bone graft materials.

### BACKGROUND

Porous calcium phosphate-based bone grafts are known in the art for use in filling bony voids or gaps in the skeletal system. Examples of such bone grafts are described in, for example, U.S. Pat. No. 6,991,803; U.S. Pat. No. 6,521,246; and U.S. Pat. No. 6,383,519, incorporated herein. Vitoss® Bone Graft Substitute (Orthovita, Inc., Malvern Pa.) is one exemplary type of such bone graft. These bone grafts incorporate a porosity structure that replicates the geometric form of bone and allows for improved biological activity and bone ingrowth at the implantation site, especially when the bone graft is combined with autogenous blood and/or bone marrow.

Such porous calcium phosphate-based bone grafts have been further modified and improved to incorporate biocompatible materials such as, for example, polymers including collagen, which imparts improved handling ability of the bone graft; and bioactive glasses, which even further enhance the biological activity of the bone graft. Examples of such materials are described in, for example, U.S. Pat. No. 7,534,451; No. 7,531,004; No. 7,189,263; and U.S. Patent App. No. 20080187571, incorporated herein. Vitoss™ BA Bioactive Bone Graft Substitute (Orthovita, Inc., Malvern, Pa.) is one exemplary type of such a bone graft incorporating bioactive glass.

While the aforementioned bone grafts promote autologous bone tissue formation, methods of improving the rate of autologous bone tissue formation are still needed. It has been reported that strontium can enhance bone formation and inhibit bone resorption. See, e.g., Bonnelye, E., et al., Dual effect of strontium ranelate: Stimulation of osteoblast differentiation and inhibition of osteoclast formation and resorption in vitro, Bone 42 (2008) 129-138; however, the effect of strontium in conjunction with a porous beta-tricalcium phosphate-based bone graft substitute has not been known.

The effect of strontium has been explored in the field of bone cements. Metallic implants can be coated with hydroxyapatite (Ca₁₀(PO₄)₆(OH)₂) to improve fixation of the implant to the hard tissues of the body and to help reduce the incidence of bone implant failure by facilitating osteointegration and stress shielding. Strontium has been incorporated into hydroxyapatite nanocrystals and these crystals have been pulse-laser deposited on etched titanium substrates and their biological activity was evaluated. This study indicated that incorporation of strontium resulted in improvement in osteoblast activation and differentiation markers. Capuccini, C., et al., Strontium-substituted hydroxyapatite coatings synthesized by pulsed-laser deposition: In vitro osteoblast and osteoclast response, Acta Biomat 4 (2008) 1885-1893.

The release of strontium from coated surfaces is diffusion controlled. Brandt et al. observed an initial burst of strontium release that was complete within the first 100 hours. As a result, strontium was only present during the first days of implantation, leading to an initial increase of de novo bone formation 3 days after implantation followed by slower bone formation afterwards. Brandt, H., et al., Cellulose 2008, 15, 275.

It has heretofore been unknown how to incorporate strontium homogeneously into highly porous bone graft materials such that improved bone formation is achieved via controlled delivery to the local implantation site. Methods and materials for the controlled release of strontium at the site of bone graft implantation are thus needed.

### SUMMARY

The present invention is directed to highly porous bone graft materials comprising calcium phosphate homogeneously blended with from about 0.1 to about 4.5 percent, by weight of the graft material, of strontium. The bone graft materials of the invention have a pore volume of at least about 70%. Preferred graft materials of the invention, in addition to having a pore volume of at least about 70%, will have interconnected macroporosity, mesoporosity, and microporosity. Methods of making these materials are also described.

### BRIEF DESCRIPTION OF THE DRAWINGS

FIG. 1A illustrates one basic form of the graft material of the present invention in cylinder form.

FIG. 1B depicts the graft material in cylindrical form 80 inserted into a bone void 83 below the femur 81 in the tibial plateau 82 within a human knee.

FIG. 2 illustrates another basic form of the present invention in strip form.

FIG. 3A illustrates one embodiment of the graft material of the present invention in semi-spherical form used as a graft containment device.

FIG. 3B depicts a semi-spherical form of the graft material 102 used to accommodate an artificial implant 103. The graft material 102 contains an acetabular cup 106, which holds a polyethylene cup 105, in this embodiment.

FIG. 4A illustrates the graft material of the present invention in disc form. FIG. 4B illustrates another embodiment of the bioactive graft material of the present invention used as a cranio-maxillofacial 76, zygomatic reconstruction 72, and mandibular implant 74.

FIG. 5 illustrates one embodiment of a graft material of the present invention described shaped into a block/wedge form and used as a tibial plateau reconstruction that is screwed, bonded, cemented, pinned, anchored, or otherwise attached in place.

FIGS. 6A and 6B illustrate synthetic resorbable defect filling bone graft materials 272 for bone restoration having mesh 270 attached to one side. FIG. 6C depicts a synthetic resorbable defect filling bone graft material block in which the mesh 270 is sandwiched between the graft material 272.

FIGS. 7A, 7B, and 7C illustrate an embodiment of the graft material of the present invention in semi-tubular form used as a long bone reinforcement sleeve. As shown in the figures, the semi-tube may have a moon cross-section with a uniform thickness (FIG. 7A); or a crescent moon cross-section with a tapered radius that comes to a point (FIG. 7B) or a tapered radius that is rounded on the edges (FIG. 7C).

FIG. 8A is an SEM image (×50 magnification) of one embodiment of the invention in morsel form showing the highly porous nature of the material. The indicator bar length is equivalent to 500 μm.

FIG. 8B is an SEM image at higher magnification (×1000 magnification) of the embodiment of FIG. 8A. The indicator bar length is equivalent to 10 μm.

FIG. 9 is an SEM image at even higher magnification (×5000 magnification) of embodiment of FIG. 8A. The indicator bar length is equivalent to 5 μm (top). An EDS spectrum of the material confirms the composition of the present invention includes calcium, phosphorus and strontium (bottom).

FIG. 10 is an SEM image (×50 magnification) of another embodiment of the present invention in strip form.

FIG. 11 is an SEM image at higher magnification (×250 magnification) of the embodiment of FIG. 10.

FIG. 12 is an SEM image (×50 magnification) of yet another embodiment of the present invention in pack form that is pliable when wetted.

FIG. 13 is an SEM image (×1,100 magnification) representing the bioactive nature of one embodiment of the present invention after submersion in simulated body fluid (SBF) for 7 days.

FIG. 14 is a faxitron image of various embodiments of the present invention showing the radiopacity of each in comparison to equivalent control materials that do not contain strontium.

FIG. 15 is a graphical depiction of strontium release from 10 cc of composite material that includes between 0.1 mol % and 5 mol % strontium-doped calcium phosphate admixed with collagen.

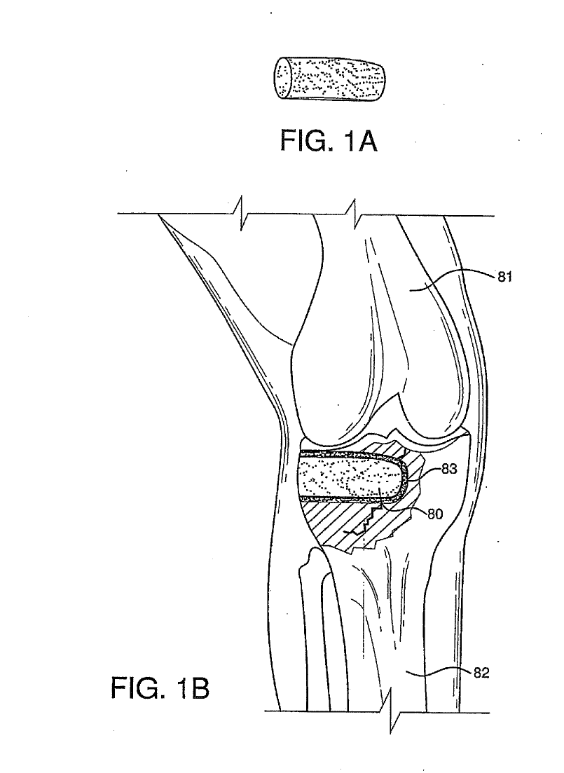

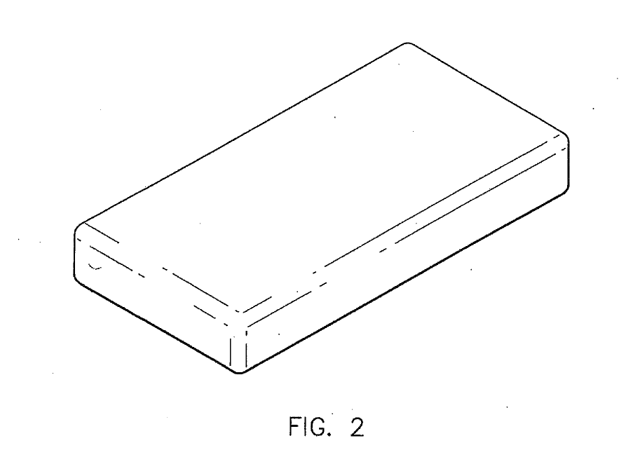

### DETAILED DESCRIPTION OF ILLUSTRATIVE EMBODIMENTS

The present invention is directed to porous bone graft materials comprising calcium phosphate and strontium. It is envisioned that incorporation of strontium into the porous calcium phosphate material will result in even more rapid bone generation at the local site of the bone graft implantation.

The bone grafts of the invention comprise highly porous calcium phosphate homogeneously blended with from about 0.1 to about 4.5 percent, by weight of the graft material, of strontium. As used herein, “homogeneously blended” means that the strontium is incorporated throughout the calcium phosphate phase of the bone graft implants rather than concentrated in a particular area of the bone graft. In preferred embodiments of the invention, the calcium phosphate and strontium are generated together in solution phase using oxidation-reduction chemistry. Thus, strontium is present throughout the bone graft material and does not reside solely at or near the surface of the bone graft material.

In addition, the bone grafts of the invention have a pore volume of at least about 70%. Zero percent pore volume refers to a fully dense material having no pores at all. One hundred percent pore volume cannot meaningfully exist since it would refer to the material being all pores, i.e., air. Persons skilled in the art understand the concept of pore volume and can easily calculate and apply it. For example, pore volume may be determined in accordance with W. D. Kingery, Introduction to Ceramics, 1960, p. 416 (Wiley, 1060), that provides a formula for determining porosity. One exemplary method of determining porosity is to use mercury intrusion porosimetry. Porosity expressed as a percentage is pore volume: Pore Volume=(1−fp)100%, where fp is fraction of theoretical density achieved.

Preferably, the bone grafts have a porosity or pore volume of between about 70% to about 95%. In certain embodiments, the bone grafts have a pore volume of between 70% and about 85%. Preferably, the bone grafts of the invention have pore volumes of at least about 75%. Also preferred are materials having pore volumes of at least about 80%-90%.

In certain embodiments of the invention, in addition to the bone grafts having pore volumes of at least about 70%, the bone grafts also comprise varying levels of porosity. In preferred embodiments, varying levels of porosity are interconnected. In exemplary embodiments of the invention, the bone grafts comprise three different porosity size ranges, herein described as macroporosity, mesoporosity, and microporosity. Preferably, the macroporosity, mesoporosity, and microporosity occur simultaneously. Within the scope of this invention, macroporosity is defined as having pore diameters greater than or equal to 100 microns. Mesoporosity is defined as having a pore diameter less than 100 microns but greater than or equal to 10 microns. Microporosity is defined as having a pore diameter less than 10 microns.

Persons skilled in the art can easily determine whether a material has each type of porosity through examination, such as through the preferred method of scanning electron microscopy. While it is certainly true that more than one or a few pores within the requisite size range are needed in order to characterize a sample as having a substantial degree of that particular form of porosity, no specific number of percentage is called for. Rather, a qualitative evaluation by persons skilled in the art shall be used to determine macroporosity, mesoporosity, and microporosity.

While the invention does not require a specific percentage for each of the three porosity size ranges described, certain percentages of each porosity size range have been found to be particularly well suited for bone graft materials. For example, in certain embodiments, the bone graft materials can be characterized as having about 10-30% of the pores within the microporosity range (i.e., less than 10 microns); about 5-15% of the pores within the mesoporosity range (i.e., 10-100 microns); and about 55-80% of the pores within the macroporosity range (i.e., 100 microns and greater). In other embodiments, the bone graft materials can be characterized as having about 15-28% of the pores within the microporosity range; about 5-12% of the pores within the mesoporosity range; and about 60-75% of the pores within the macroporosity range. In still other embodiments, the bone graft materials can be characterized as having about 10-25% of the pores within the microporosity range; about 50-70% of the pores within the mesoporosity range; and about 10-30% of the pores within the macroporosity range.

Various forms of calcium phosphate are known in the art to be useful in orthopedic applications. Table 1 lists a sampling of the most commonly used calcium phosphates, including the calcium:phosphorous ratio present in each. It is envisioned that any of these calcium phosphates can be modified, in view of the procedures set forth herein or known in the art, to include strontium according to the invention.

TABLE 1

| Ca/P | Formula | Name |
|---|---|---|
| 0.5 | Ca(H₂PO₄)₂•H₂O | Monocalcium Phosphate Monohydrate |
| 1.0 | CaHPO₄•2H₂O | Hydrated Calcium Phosphate/Brushite |
| 1.0 | CaHPO₄ | Anhydrous Calcium Phosphate/Monetite |
| 1.33 | Ca₈H₂(PO₄)₆•5H₂O | Octacalcium Phosphate |
| 1.5 | Ca₃(PO₄)₂ | Tricalcium Phosphate (Whitlockite) |
| 1.67 | Ca₁₀(PO₄)₆(OH)₂ | Hydroxylapatite |
| 2.0 | CaO•Ca₃(PO₄)₂ | Tetracalcium Phosphate/Hilgenstockite |

In preferred embodiments of the invention, the calcium phosphate is tricalcium phosphate with a calcium:phosphorous ratio of 1.5:1. More preferably, the calcium phosphate of the present invention is beta tricalcium phosphate (β-tricalcium phosphate or β-TCP).

In addition to including homogenously blended calcium phosphate and strontium, the bone graft materials of the invention may also include biocompatible polymers, for example, collagen. The incorporation of such polymers improves, for example, the handling of the bone graft materials of the invention. Preferably, the biocompatible polymer is homogenously blended throughout the bone graft material. In preferred embodiments, the bone graft materials of the invention comprise from about 10% to about 40%, by weight of the graft, of the biocompatible polymer. Exemplary embodiments comprise about 10-25%, by weight of the graft, of biocompatible polymer.

Bone graft materials of the invention may also include a filler. Preferably, the filler is an inorganic material, for example, barium glass, barium-boroaluminosilicate glass, silica, 45S5 glasses, bioactive glass ceramics, glass-ceramics, bioactive synthetic Combeite glass-ceramic, or combinations thereof. In preferred embodiments, the filler is bioactive. As used herein, “bioactive” relates to the chemical formation of a calcium phosphate layer via ion exchange between surrounding fluid and the graft material. “Bioactive” can also relate to materials that elicit a reaction which leads to bone formation or attachment into or adjacent to grafts or to bone formation of apposition directly to the grafts usually without intervening fibrous tissue. Bone graft materials of the invention preferably include up to about 35%, by weight of the graft, of filler. Preferably, the materials of the invention comprise about 5-35%, by weight of the graft, of filler.

In certain embodiments, the bone graft materials of the invention include both a biocompatible polymer and a bioactive filler. Preferably, the biocompatible polymer is collagen and the bioactive filler is 45S5 glass or Combeite glass-ceramic. Combeite is a mineral having the chemical composition Na₄Ca₃Si₆O₁₆(OH)₂. It is preferred that the Combeite glass-ceramic particles which form some or all of the bioactive glass component of the present invention comprise at least about 2% by volume of Combeite crystallites. Combeite glass-ceramic particles containing higher percentages of crystallites are more preferred and volume percentage from about 5% to about 50% of crystallites are particularly desired. It will be appreciated that the Combeite glass-ceramic particles of the present invention are heterogeneous in that they comprise a glassy, amorphous structure having crystallites or regions of Combeite crystallinity dispersed throughout the material.

In one embodiment of the present invention, the heterogeneous particles of Combeite glass-ceramic have average particle sizes of from less than about 150 μm. In other embodiments of the present invention, two particular Combeite glass-ceramic average particle size ranges have been found to be preferred, in combination, when practiced with the present invention. The first range is less than or equal to about 53 μm. The second average particle size range is from about 90 μm to about 150 μm. The combination of these two ranges practiced together with the present invention is referred to as a bimodal particle size range and/or bimodal particle size distribution.

In certain instances, it may be convenient to calculate the amount of strontium in the materials of the invention based on either mol % or weight %. It has been determined that 1 mol % is approximately equal to about 0.85 weight % of the strontium-doped calcium phosphate; 2 mol % is approximately equal to about 1.69 weight % of the strontium-doped calcium phosphate; 3 mol % is approximately equal to about 2.54 weight % of the strontium-doped calcium phosphate; 4 mol % is approximately equal to about 3.39 weight % of the strontium-doped calcium phosphate; and 5 mol % is approximately equal to about 4.24 weight % of the strontium-doped calcium phosphate.

In preferred embodiments of the present invention in which the strontium-doped calcium phosphate is incorporated into composite matrices of collagen or matrices of collagen and bioactive glass, the mass percent (%) strontium of the total composite will vary depending upon the amount of strontium-doped calcium phosphate included in the composite. For instance, in some embodiments in which 1 mol % strontium-doped calcium phosphate comprises 80% of the composite, the mass % strontium of the composite material will vary from about 0.5% to about 0.7%; while in embodiments in which 2 mol % strontium-doped calcium phosphate comprises 80% of the composite, the mass % strontium of the composite material will vary from about 1.0% to about 1.5%. In other embodiments in which 3 mol % strontium-doped calcium phosphate comprises 80% of the composite, the mass % strontium of the composite will vary from about 1.6% to about 2.1%; while in embodiments in which 4 mol % strontium-doped calcium phosphate comprises 80% of the composite, the mass % strontium of the composite will vary from about 2.15% to about 2.7%. Further, in certain embodiments in which 5 mol % strontium-doped calcium phosphate comprises 80% of the composite, the mass % strontium of the composite will vary from about 2.7% to about 3.5%.

In certain embodiments, from about 0.1% to about 4.5% by weight of the graft material of the present invention is strontium.

In general, the materials of the invention are prepared by providing an aqueous solution comprising a calcium source, for example calcium nitrate, and a strontium source, for example strontium nitrate, strontium chloride or strontium malonate, with the strontium present in about 0.1 mol % to 5.0 mol %, based on the moles of divalent salts present in the solution. The solution further comprises at least one phosphate-containing anion oxidizable by the nitrate anion. The solution is imbibed onto a porous substrate, the porous substrate having a pore volume of at least about 70% and optionally having interconnected macroporosity, mesoporosity, and microporosity.

Upon heating of the imbibed porous substrate, the components of the solution undergo an oxidation-reduction reaction between the nitrate anions and the phosphate containing anions to form a solid. The resulting solid contains a homogenous blend of calcium phosphate and strontium phosphate. Further heating results in the incineration of the porous substrate. Heating/sintering of the material can be accomplished at a temperature of at least about 800° C. up to 1300° C. In a preferred embodiment, the final heating of the material is conducted at temperatures of about 1100° C.

It should be understood that due to this unique solution based methodology of making the present invention, the materials of the invention can be homogeneously prepared in any predetermined form, for example, blocks or strips, by selecting a correspondingly-shaped porous substrate. The materials of the invention can also be shaped into any anatomical shape, as required by the physician, to repair a given anatomical defect. Morsels can be created by pulverization of the shaped material. Materials made with biocompatible polymers such as collagen can be cut and shaped to fit the defect.

It should be recognized that the source of the strontium for this invention is not limited to the aforementioned examples. Strontium is readily available in other forms including but not limited to acetate, bromide, citrate, iodide, oxalate and sulfate salts. Strontium cations may also be generated in-situ by a reaction between strontium metal and water. Other embodiments of this invention may utilize strontium containing materials or nanomaterials as a source of strontium.

Non-limiting examples of strontium sources that may be utilized in the present invention include strontium acetate hydrate, strontium bromide hexahydrate, anhydrous strontium bromide, strontium carbonate, strontium chloride hexahydrate, anhydrous strontium chloride, strontium citrate, strontium malonate, strontium nitrate, strontium oxalate, strontium ranelate and strontium sulfate.

The use of a solution based chemistry to prepare the material of the present invention is particularly desired to allow shaped bodies as described herein to be easily made via the use of substrates and sponges with the desired homogeneity, porosity and shape characteristics. It may be preferable that a support material be incorporated into the bone graft materials of the invention. Certain preferred support materials include metal (e.g., stainless steel or titanium) mesh, rods, plates, and the like.

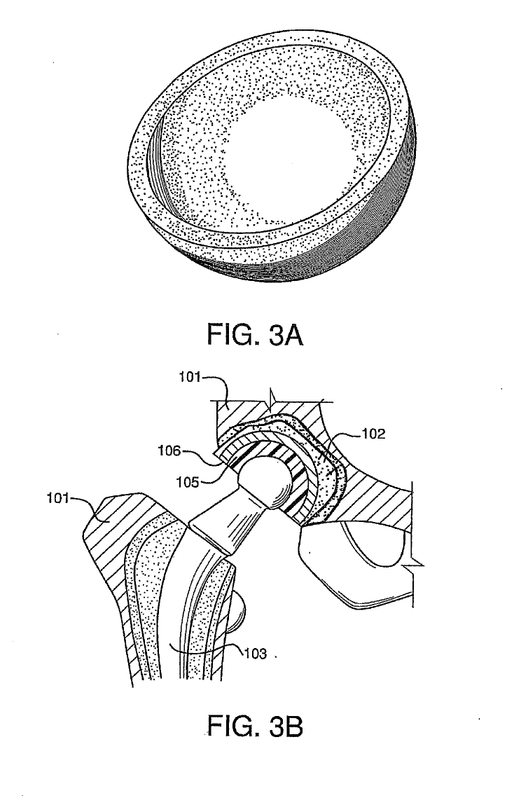

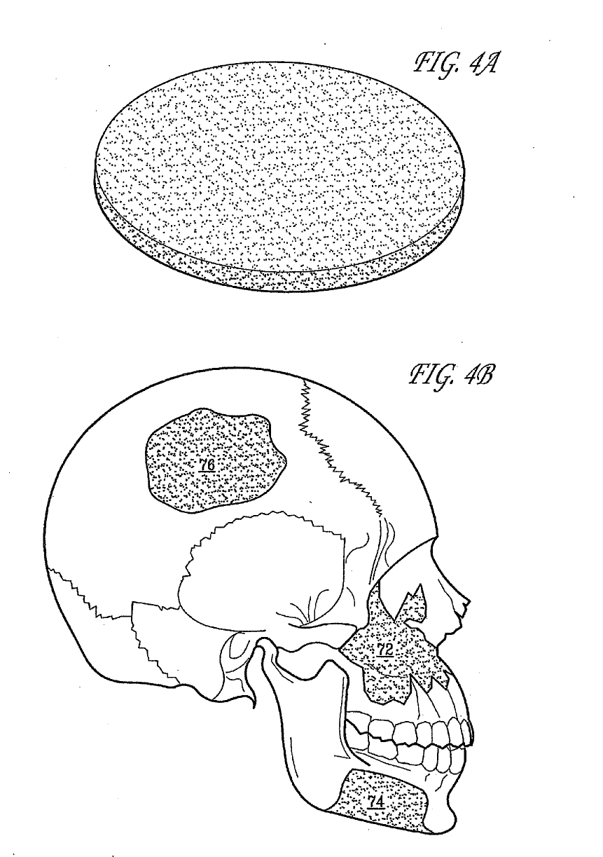

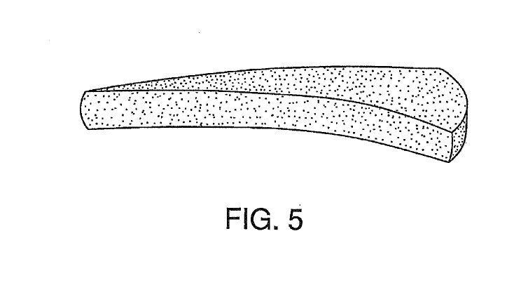

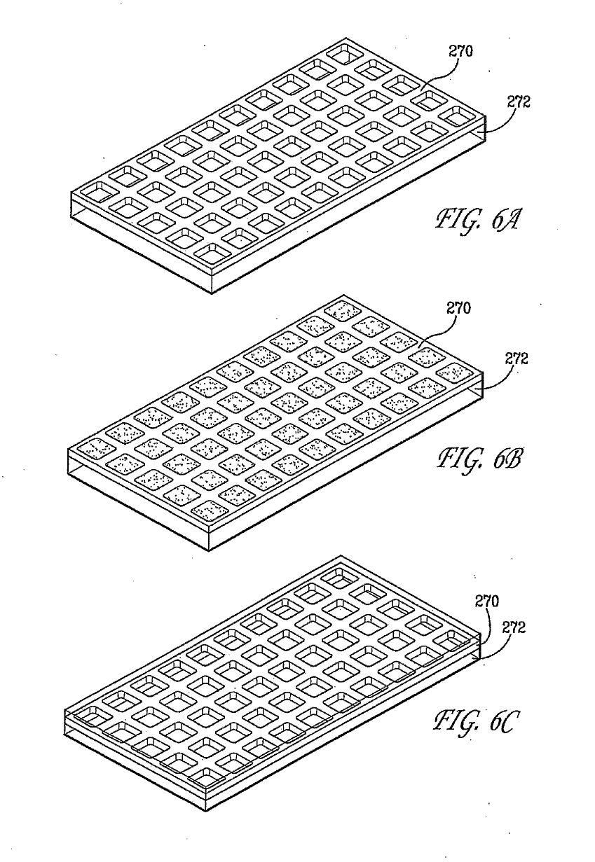

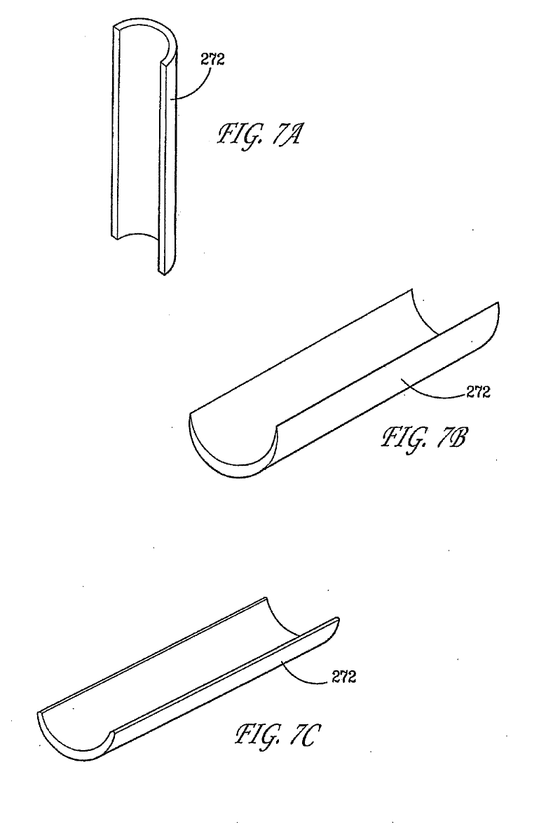

Due to the wide range of applications for the embodiments of the present invention, it should be understood that the present invention graft material may be used in a variety of clinical applications. For instance, blocks and cylinders of the present invention may find utility in bone void filling and filling of interbody fusion devices; wedge shaped devices of the present invention may find utility in high tibial osteotomies; and strips may find utility in cranial defect repairs. Of particular interest, may be the use of some of the graft materials as semi-spherical (FIG. 3A), semi-tubular (FIGS. 7A-7C) or disc-shaped (FIG. 4A) strips for graft containment devices. An embodiment of the semi-spherical form 102 in use is depicted in FIG. 3B.

It will be appreciated that these shapes are not intended to limit the scope of the invention as modifications to these shapes may occur to fulfill the needs of one skilled in the art. The benefits of the graft containment materials that, for instance, may be used in acetabular reconstruction made from the present invention are several-fold. The graft materials may act as both a barrier to prevent migration of other implants or graft materials and serves as an osteoconductive resorbable bone graft capable of promoting bone formation. The graft containment device may be relatively non-load-bearing, or partially load-bearing, or may be reinforced to be fully load-bearing as described below. Depending on the form, the graft material has barrier properties because it maintains its structural integrity.

In applications requiring graft materials with load-bearing capabilities, the graft materials of the present invention may have meshes or plates affixed or incorporated. The meshes or plates may be of metal, such as titanium or stainless steel, or of a polymer or composite polymer such as polyetheretherketone (PEEK), or nitinol. As depicted in FIGS. 6A and 6B, a metallic mesh 270 may be placed to one side of the bone graft material 272 to add strength and load-bearing properties to the implant. In FIG. 6A, the mesh plate 270 sits affixed to one surface of the graft material 272. In FIG. 6B, the mesh plate 270 penetrates one surface of the graft material 272 with one side of mesh exposed on top. In FIG. 6C, the mesh plate 270 is immersed more deeply than in FIG. 6B within the graft material 272. FIGS. 7A-7C depict another embodiment of the graft material 272 in semi-tubular form. A mesh may be affixed to a surface for further support in long bone reinforcement. Due to the unique properties of the present invention graft material, the mesh may be affixed in the body using sutures, staples, screws, cerclage wire or the like.

One skilled in the art may place the mesh in any location necessary for a selected procedure in a selected bodily void. For instance, a composite of mesh and graft material could be used in a craniomaxillofacial skull defect with the more pliable graft surface being placed in closer proximity to the brain and the more resilient mesh surface mating with the resilient cortical bone of the skull. In this manner, the mesh or plate may be affixed to one side of the graft material. Alternatively, the mesh or plate may be affixed to both sides of the graft material in sandwich fashion. Likewise, graft material could be affixed to both sides of the mesh or plate. In some embodiments, the mesh may be immersed within the graft material. The meshes may be flat or may be shaped to outline the graft material such as in a semi-spherical, semi-tubular, or custom form.

In accordance with the present invention, another embodiment provides a bone graft for long bone reinforcement semi-tubular shape, or sleeve, the entire graft having interconnected macro-, meso-, and microporosity. A mesh may be affixed to the surface of the sleeve or may be immersed in the sleeve. The mesh may be made of titanium, stainless steel, nitinol, a composite polymer, or polyetheretherketone. The cross-section of the sleeve may be in the shape of a crescent-shaped moon (FIG. 7B).

In other embodiments, there is a graft for the restoration of bone in the form of a shaped body, the material of the graft having interconnected macro-, meso-, and microporosity; the body shape being selected to conform generally to a mammalian, anatomical bone structure. The shapes will vary depending on the area of the body being repaired. Some basic shapes may be a disk, semi-sphere, semi-tubular, or torus. In some embodiments, the shape will conform generally to the acetabulum.

Other graft materials of the present invention having load-bearing capabilities may be open framed, such that the bone graft material is embedded in the central opening of the frame. The frame may be made of a metal such as titanium or of a load-bearing composite such as PEEK or a composite or some form of poly-lactic acid (PLA).

The bone graft materials of the invention have a strontium dissolution profile, i.e., strontium release rate, heretofore not previously reported. The materials of the invention provide for a sustained release of strontium over time in vivo, for example, over at least three weeks, preferably over at least 12-24 weeks. As used herein, “sustained” relates to continued release of strontium from the bone graft material over a given period of time. It is noted that while more or less strontium may be released from the bone graft material at different times, at least some quantity of strontium will be released over the entire time period.

The porosity of the materials of the present invention, coupled with the solution-based blending of the calcium phosphate and strontium surprisingly provides for the formation of stable calcium phosphate materials in which strontium ions are homogeneously incorporated resulting in unique strontium release profiles. A large percentage of the strontium continues to remain bound within the crystal structure of the material under in-vitro conditions. Thus, the majority of strontium release takes place through a cell-mediated resorption process. For example, cellular phagocytosis of the material creates a local acidic environment resulting in strontium release from the material, as measured in-vitro via inductively coupled plasma mass spectrometry (ICP). It is envisioned that this process will provide for controlled local delivery of strontium ions to the implantation site over the course of material degradation and bone remodeling. As such, the local delivery profile will be controlled by the resorption profile of the bone graft of the invention.

Non-strontium-containing porous calcium phosphate bone grafts are known to exhibit about 50-90% resorption by 12 weeks and to approach 100% resorption by 52 weeks. It is anticipated that a similar resorption profile would be exhibited by the materials of the present invention.

In order to estimate the amount of strontium release that can be anticipated in-vivo, the resorption rate of Vitoss® Bone Graft Substitute, an exemplary form of porous calcium phosphate containing macroporosity, mesoporosity, and microporosity (Orthovita, Inc., Malvern, Pa.) was used as a model for the strontium-containing materials of the invention. It has been demonstrated clinically that Vitoss Bone Graft Substitute will achieve approximately 50-90% resorption after 12 weeks and 90-100% by 24 weeks. See, e.g., Clineff, T., et al., Analytical Technique for Quantification of Selected Resorbable Calcium Phosphate Bone Void Fillers with the Use of Polarized-Light Microscopy, J Biomed Mater Res Part B: Applied Biomater 72B: (2005) 125-130; and Erbe, E., et al., Potential of an ultraporous β-tricalcium phosphate synthetic cancellous bone void filler and bone marrow aspirate composite graft, Eur Spine J 10: (2001) S141-S146.

Table 2 below displays the controlled delivery of strontium in mg per day for 1 g of Vitoss Bone Graft Substitute with strontium substitution of between 1 and 5 mol % assuming a range of 50-90% resorption rate.

TABLE 2
Strontium release (mg per day) for 1 g of Vitoss Bone Graft
Substitute “doped” with Strontium (Sr)

| | | | | mg Sr per | mg Sr per |
|---|---|---|---|---|---|
| Mol % Sr | mgSr/g | | | day through | day from |
| Substitution | VT | 50% | 90% | 12 weeks | 12-24 weeks |
| 5% | 42.37 | 21.19 | 38.13 | 0.25-0.45 | 0.05-0.25 |
| 4% | 33.90 | 17.00 | 30.51 | 0.20-0.36 | 0.04-0.20 |
| 3% | 25.42 | 12.71 | 22.88 | 0.15-0.27 | 0.03-0.15 |
| 2% | 16.95 | 8.48 | 15.25 | 0.10-0.18 | 0.02-0.10 |
| 1% | 8.47 | 4.24 | 7.63 | 0.05-0.09 | 0.01-0.05 |

Vitoss® Foam is another exemplary form of porous calcium phosphate containing macroporosity, mesoporosity, and microporosity. Vitoss Foam differs from Vitoss Bone Graft Substitute, primarily in that Vitoss Foam further includes collagen. Table 3 displays the controlled delivery of strontium in mg per day for 10 cc of Vitoss Foam (Orthovita, Inc., Malvern, Pa.) with strontium substitution of between 1 and 5 mol %. The resorption is assumed to occur at a predictable rate over the course of the first 12 weeks.

TABLE 3
Strontium release (ppm per day) for a 10 cc Vitoss
Foam* unit “doped” with Strontium (Sr)

| | | | | ppm Sr per | ppm Sr per |
|---|---|---|---|---|---|
| Mol % Sr | ppmSr/10 | | | day through | day from |
| Substitution | cc strip | 50% | 90% | 12 weeks | 12-24 weeks |
| 5% | 13559 | 6780 | 12203 | 81-145 | 16-81 |
| 4% | 10847 | 5424 | 9763 | 65-116 | 13-65 |
| 3% | 8135 | 4068 | 7322 | 48-87 | 10-48 |
| 2% | 5424 | 2712 | 4881 | 32-58 | 6-32 |
| 1% | 2712 | 1356 | 2441 | 16-29 | 3-16 |

*Assumptions include a total weight of 4 g for a 10 cc sample, and 80% of the weight of the Vitoss Foam being a strontium-doped calcium phosphate component (3.2 g). To obtain ppm levels, the volume of fluid added to the Vitoss Foam is assumed to be 10 cc.

Cell culture studies have shown that the presence of strontium ions at similar concentrations has positive effects on osteoblasts. Barbara, A., et al., Normal matrix mineralization induced by strontium ranelate in MC3T3-E1 osteogenic cells, Metabolism 53 (2004): 532-7; Bonnelye, E., et al., Dual effect of strontium ranelate: Stimulation of osteoblast differentiation and inhibition of osteoclast formation and resorption in vitro, Bone 42 (2008): 129-138; Cappuccini, C., et al., Strontium-substituted hydroxyapatite coatings synthesized by pulsed-laser deposition: In vitro osteoblast and osteoclast response, Acta Biomat 4 (2008): 1885-1893.

Cell culture studies with strontium doped media have shown positive effects on the proliferation and alkaline phosphatase activity of osteoblast-like cells. Alkaline phosphatase is a biochemical marker of bone formation.

Clinically, the positive effect of strontium has been noted in two separate phase II dose ranging studies, PREVOS (PREVention Of early postmenopausal bone loss by strontium ranelate) and STRATOS (STRontium Administration for Treatment of Osteoporosis). The PREVOS study showed a 5.53% increase in lumbar bone mineral density with oral administration of 1 g strontium ranelate/day compared to baseline and was effective in preventing bone loss. The serum strontium levels achieved with 1 g/day in the PREVOS study was measured to be 6.13 ppm after 24 months of treatment. The STRATOS study showed an annual increase of 7.3% per year for lumbar bone mineral density at a dose of 2 g strontium ranelate per day. Meunier, P. J., et al., Osteoporosis Int. 14 (2003): S56-S65. This dose resulted in a serum strontium level of 10.56 ppm. Marie, P. J., Osteoporosis Int. 19 (2008):1813. However, unlike these reported clinical studies, the present invention provides for a local method of delivery of strontium, rather than systemic delivery.

Thus, the bone graft materials of the invention comprising calcium phosphate doped with strontium will demonstrate more rapid bone formation than bone graft materials previously used in the art.

### EXAMPLES

### Example 1

### Solution-Based Synthesis of Strontium Doped Calcium Phosphate

Once the desired Sr/(Sr+Ca) was selected (Table 4 sets forth proportions of reagents for preferred embodiments), the requisite amount of strontium nitrate and water was added to the reaction vessel. The strontium nitrate was dissolved at about 67° C. and the requisite amount of calcium nitrate was added. The resulting solution was divided into three portions. Hypophosphorous acid was added to one of the three portions, and the mixture was stirred for 15 minutes. The resulting solution was then used in the foregoing examples to prepare materials of the invention.

TABLE 4

| | Ca(NO₃)₂•4H₂O | Sr(NO₃)₂ | Total H₂O |
|---|---|---|---|
| Sr/(Sr + Ca) | (g) | (g) | (g) |
| 0.00 | 11000.00 | 0.00 | 3446.00 |
| 0.01 | 10890.00 | 98.58 | 3479.57 |
| 0.02 | 10780.00 | 197.16 | 3513.13 |
| 0.03 | 10670.00 | 295.74 | 3546.70 |
| 0.04 | 10560.00 | 394.31 | 3580.27 |
| 0.05 | 10450.00 | 492.89 | 3613.83 |

### Example 2

### Strontium-Doped Calcium Phosphate-Block (SrCPBlock)

A sponge block was immersed in the solution of Example 1 and kneaded to fully imbibe the reactant solution into the sponge. Energy was applied to the sponge block, thoroughly saturated with reactant solution, and after several seconds, a reaction commenced at the surface of the sponge with the evolution of red-brown fumes characteristic of NO_{x(g)}. The sponge was then heated to about 1100° C. to produce the finished material. The resulting material was then used in the foregoing examples alone and in combination with other materials as described below.

### Example 3

### ICP Testing of 5 Mol % Strontium-Doped Calcium Phosphate

The present invention strontium-doped β-tricalcium phosphate (Ca₃(PO₄)₂) material (5 mol %) was tested using inductively-coupled plasma-optical emission spectroscopy (ICP-OES or ICP). ICP testing indicated near ideal strontium incorporation into the material (Table 5). The material showed a greater than 95% match to β-tricalcium phosphate on x-ray diffraction, indicating β-tricalcium phosphate as the primary phase. When the actual mass % values were converted into mol values for Ca, Sr and P using a sample mass of 71.72 mg, the Ca+Sr/P ratio was calculated to be 1.493, or about 1.5, the desired calcium:phosphorous ratio.

TABLE 5
ICP Testing of 5 mol % Strontium-Doped Calcium Phosphate

| Element Tested | 5% |
|---|---|
| Theoretical mass % Sr | 4.24 |
| Actual mass % Sr | 4.04 |
| Theoretical mass % Ca | 36.83 |
| Actual mass % Ca | 36.75 |
| Theoretical mass % P | 19.97 |
| Actual mass % P | 20.87 |

### Example 4

### Porosity of 5 Mol % Strontium-Doped Calcium Phosphate Block Material

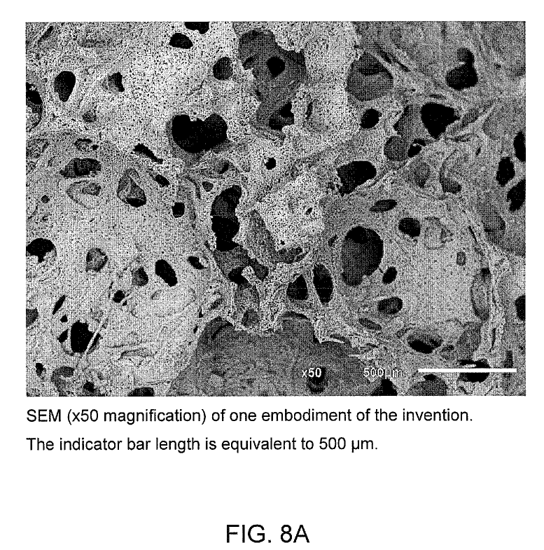

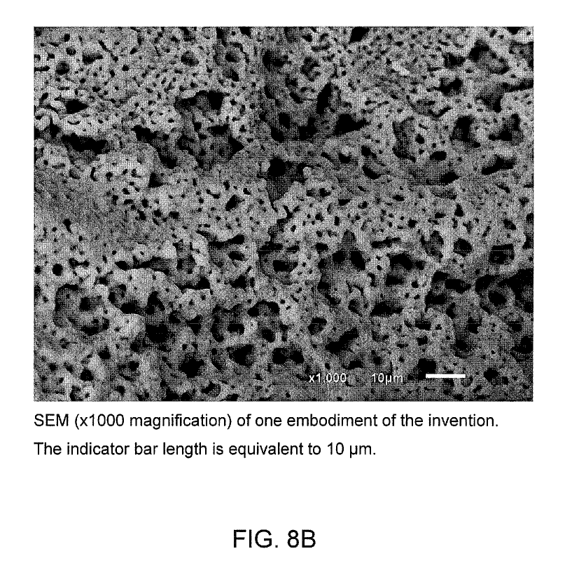

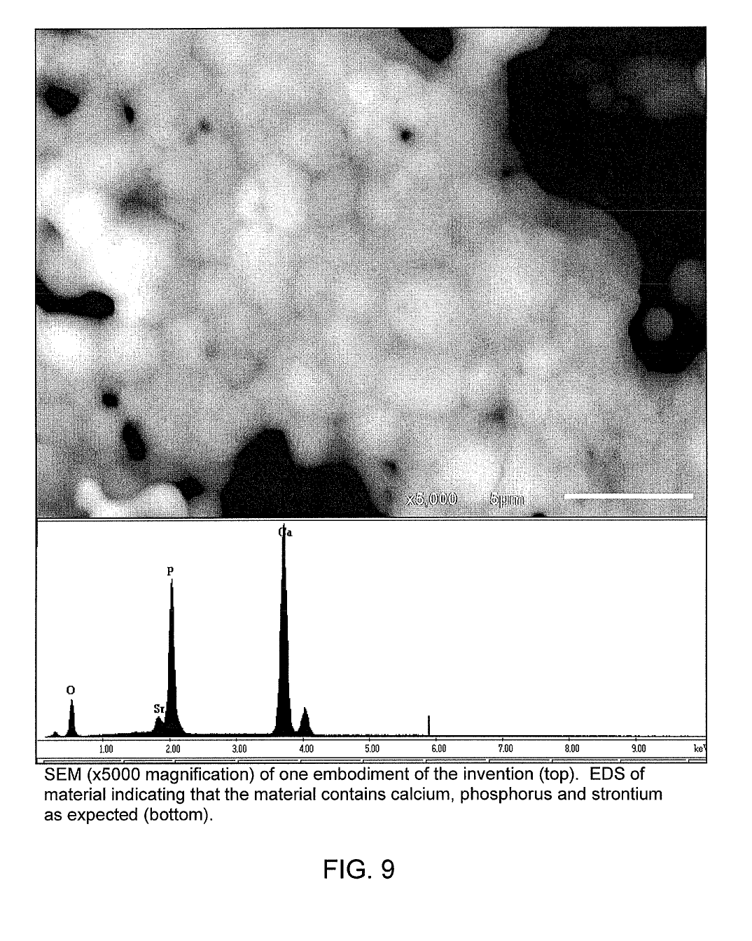

The porous character of bone graft substitute containing 5 mol % strontium-doped calcium phosphate having macro-, meso- and microporosity was examined using mercury intrusion porosimetry. The porosity profile is shown in Table 6. Scanning electron microscopy also confirmed this porous morphology (FIGS. 8A, 8B, and 9).

TABLE 6
Porosity Profile of 5 mol % Strontium-Doped Calcium Phosphate

| Pore Size | |
|---|---|
| Diameter (μm) | Frequency |
| <10 | 24.7% |
| 10-100 | 9.4% |
| 100-1000 | 65.9% |

The total porosity of this sample was about 84.4%.

### Example 5

### Strontium-Doped Calcium Phosphate with Combeite and Collagen

### SrCP Bioactive Foam

The block material characterized in Example 3 was crushed and sieved to obtain morsels in the size range of 1-2 mm. The morsels were added to a fibrous collagen slurry in a wet processing room and the resultant slurry was further mixed with bioactive Combeite glass-ceramic and casted/molded into various shapes in a cleanroom. The shapes were freeze-dried and crosslinked using dehydrothermal (DHT) treatment to produce resultant bone graft material shaped products. The samples had a final ratio of about 70% strontium-doped calcium phosphate: about 10% Combeite glass-ceramic and about 20% collagen. The attributes of the material in strip and form were examined using scanning electron microscopy (SEM). The clinical handling, wettability and compression resistance of the material were also evaluated. Finally, bioactivity of the samples was performed.

Scanning Electron Microscopy

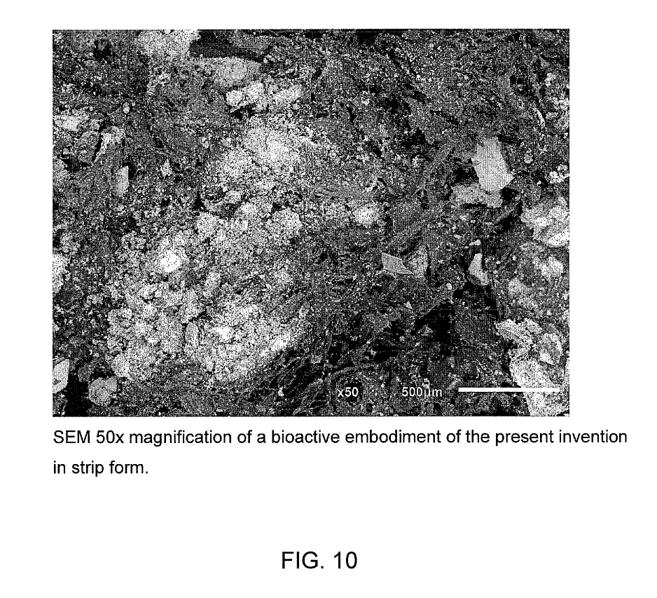

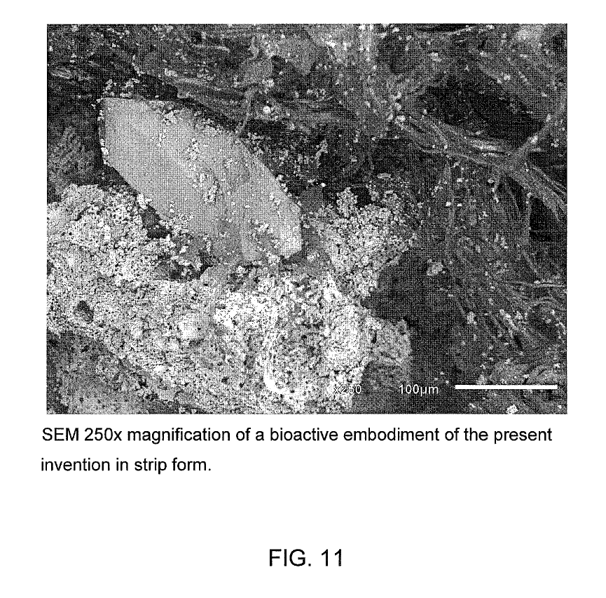

SEM imaging of the composite graft material containing 5 mol % strontium doped calcium phosphate, Combeite bioactive glass-ceramic and collagen (in strip form) showed good distribution of the strontium-doped calcium phosphate morsels and Combeite particles within the collagen network. Processing did not cause any apparent changes in the Combeite or the strontium-doped calcium phosphate morsels (FIGS. 10, 11).

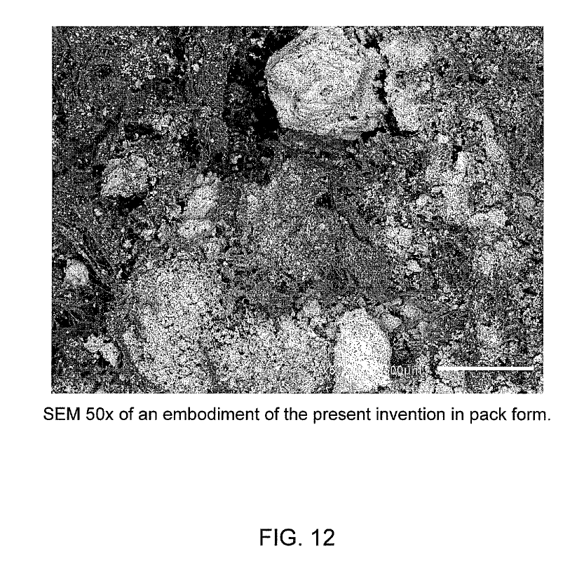

SEM imaging of the composite graft material containing 5 mol % strontium-doped calcium phosphate, Combeite bioactive glass-ceramic and collagen in pliable, putty form (“SrCP Bioactive Foam Pack”—samples had a final ratio of about 64% strontium-doped calcium phosphate: about 20% Combeite glass-ceramic and about 16% collagen) was similar with good distribution of the strontium doped calcium phosphate morsels within the collagen and no changes in the Combeite or the strontium-doped calcium phosphate morsels (FIG. 12).

Clinical Handling

Dry test materials of the present invention (SrCP Bioactive Foam, strip form) were imbibed with saline and were manually surveyed for flexibility, structural integrity, and handling properties. All tested samples demonstrated the ability to wick fluid, and all were flexible upon wetting.

Wettability and Compression Resistance

Dry test materials of the present invention (SrCP Bioactive Foam, strip form with dimensions: 25×25×4 mm) were weighed then dipped (“soaked”) in saline for 2 minutes. The weight of the soaked sample was measured and recorded as the wet weight. The soaked samples were then placed on a wire mesh suspended above a weigh boat and a 500 g mass was placed on the samples for about 10 seconds. The samples were then weighed again to assess retention under compressive load (Table 7).

TABLE 7

| | Dry | Wet | Mass | Wet (Hydrated) | |
|---|---|---|---|---|---|
| Sample | Weight | (Hydrated) | Increase | Weight After | % Fluid |
| No. | (g) | Weight (g) | (%) | Compression (g) | Retention |
| 1 | 1.0475 | 3.9384 | 276.0% | 3.7105 | 94.2% |
| 2 | 0.9982 | 3.5277 | 253.5% | 3.3502 | 95.0% |
| 3 | 0.9194 | 3.6537 | 297.4% | 3.4637 | 94.8% |
| 4 | 1.0254 | 3.8888 | 279.2% | 3.6685 | 94.3% |
| AVG | | | 276.5% | | 94.6% |

In-Vitro Bioactivity

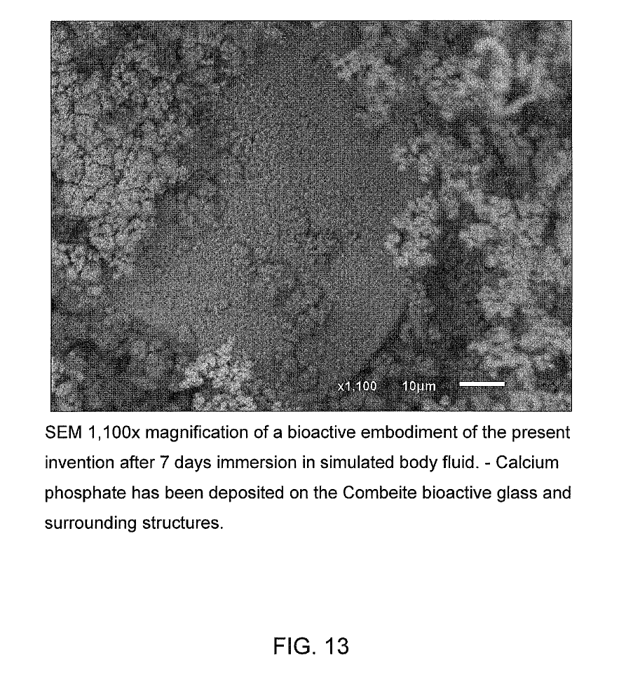

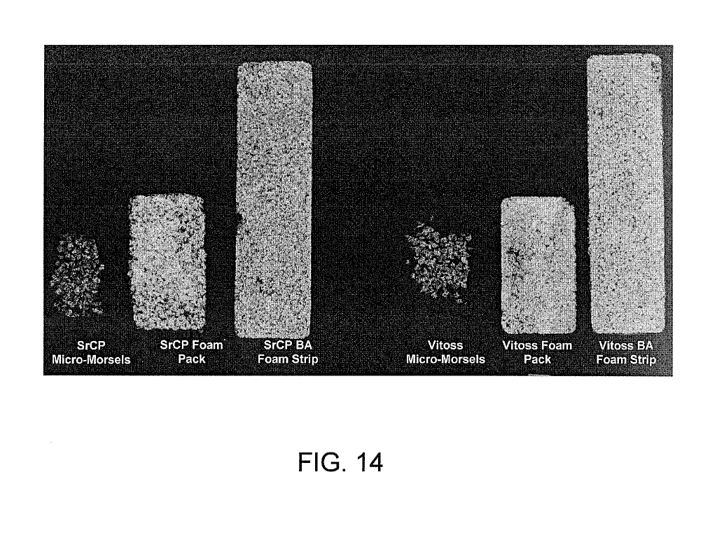

In vitro bioactivity studies were performed with the test materials of the present invention (SrCP Bioactive Foam, strip form with dimensions: 6×4×12 mm) using the method of Kokubo, How useful is SBF in predicting in vivo bone bioactivity, Biomaterials (2006) 27:2907-2915. Samples were suspended in simulated body fluid at 37° C. for 7 days. After immersion in SBF for 7 days, the formation of a significant amount of calcium phosphate can be observed on the SrCP bioactive (FIG. 13).

### Example 6

### Wash-Away Resistance of Strontium-Doped Calcium Phosphate with Collagen in Pack Form

About 5 cc of bone graft material comprised of 5 mol % strontium-doped calcium phosphate and collagen was hydrated with about 4.5 cc of colored saline, and was kneaded to a moldable putty-like consistency. To test for wash-away resistance, the putty-like composite material prepared as described was rolled into a ball and was placed in a weigh boat filled with saline solution for about 2 minutes. The material remained a continuous object and did not swell in size substantially greater than its original dimensions.

### Example 7

### Radiopacity of 5 Mol % Strontium-Doped Calcium Phosphate (SrCP), 5 Mol % Strontium-Doped Calcium Phosphate with Combeite and Collagen in Strip Form (SrCP Bioactive Foam Strip), 5 Mol % Strontium-Doped Calcium Phosphate with Combeite and Collagen in Pack Form (SrCP Bioactive Foam Pack)

A faxitron high-resolution x-ray was taken of SrCP morsels (1-2 mm), SrCP Bioactive Foam Strip and SrCP Bioactive Foam Pack in comparison to the undoped calcium phosphate equivalents of each embodiment as shown in FIG. 14. The radiopacity of each of the SrCP embodiments was similar to that of its undoped calcium phosphate equivalent.

### Example 8

### Dissolution Study of Strontium-Doped Calcium Phosphate Morsels (1-2 mm)

Dissolution of strontium doped calcium phosphate morsels was measured by ICP at different time periods (procedure—4 g/20 ml morsels at 37° C. in pH6 aqueous solution). Four grams of 5 mol % Sr-doped morsels were immersed in 20 mL buffer solution. Four different samples were prepared and pulled at 1 day, 3 days, 7 days and 14 days respectively. The pH of the 7 day samples was adjusted to pH 6.0 at day 3 and the pH of the 14 day samples was adjusted to 6.0 at the 3-day, 7-day and 10-day time points. The data are presented in Table 8.

TABLE 8
Evolution of strontium and calcium from
undoped and doped samples respectively

| | | Sr | Ca | Final | |
|---|---|---|---|---|---|
| Sample | Time | (ppm) | (ppm) | Volume | |
| Undoped | 1 | — | 598 | 20 | mL |
| | Day | | | | |
| Undoped | 3 | — | 589 | 20 | mL |
| | Day | | | | |
| Undoped | 7 | — | 660 | 22.5 | mL |
| | Day | | | | |
| Undoped | 14 | — | 995.9 | 22.8 | mL |
| | Day | | | | |
| Sr | 1 | 57.1 | 540 | 20 | mL |
| Doped | Day | | | | |
| Sr | 3 | 59.1 | 548 | 20 | mL |
| Doped | Day | | | | |
| Sr | 7 | 66 | 636 | 22.5 | mL |
| Doped | Day | | | | |
| Sr | 14 | 103 | 961.4 | 22.8 | mL |

| | |
|---|---|
| Doped | Day |

### Example 9

### Projected In-Vivo Release of Strontium from Strontium-Doped Calcium Phosphate Materials

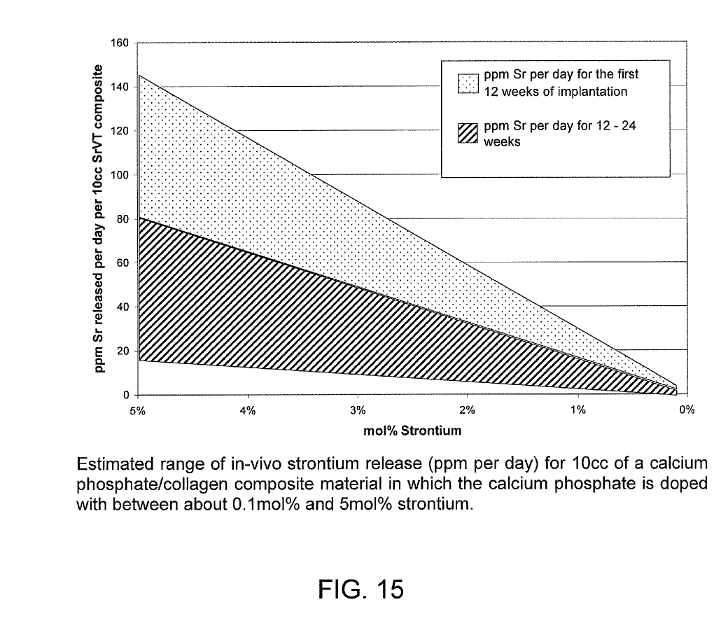

In order to estimate the amount of in-vivo strontium release anticipated, the resorption rate of Vitoss® Bone Graft Substitute (Orthovita, Inc., Malvern, Pa.) was used as a model for the strontium-containing materials of the invention. Using a clinically appropriate resorption rate range of between 50-90% based on the form of the material being placed in the defect site, the in-vivo release of strontium from 10 cc of material doped with varying amounts of strontium ranges from about 3 ppm to about 145 ppm (Sr) per day for the first 12 weeks and from about 1 ppm to about 81 ppm (Sr) per day for the 12-24 week time period post-implantation as depicted in FIG. 15.

Although illustrated and described above with reference to certain specific embodiments and examples, the present invention is nevertheless not intended to be limited to the details shown. Rather, various modifications may be made in the details within the scope and range of equivalents of the claims and without departing from the spirit of the invention. It is expressly intended, for example, that all ranges broadly recited in this document include within their scope all narrower ranges which fall within the broader ranges.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A bone graft material comprising calcium phosphate homogeneously blended with from 0.1 to about 4.5 percent, by weight of the graft material, of strontium; the bone graft material having a pore volume of at least about 70%.

2. The bone graft material of claim 1, having macroporosity, mesoporosity, and microporosity.

3. The bone graft material of claim 1, wherein at least 90% of the calcium phosphate is β-tricalcium phosphate.

4. The bone graft material of claim 1, wherein the pore volume is at least about 75%.

5. The bone graft material of claim 1, further comprising collagen.

6. The bone graft material of claim 1, further comprising bioactive glass.

7. The bone graft material of claim 1, further comprising collagen and bioactive glass.

8. The bone graft material of claim 1 in the form of a block, a strip, or morsels.

9. A method of preparing a bone graft comprising
providing an aqueous solution comprising calcium nitrate, a strontium source providing from 0.1 to 5.0 mol % of strontium, based on the moles of divalent salts present in the solution and at least one phosphite-containing anion oxidizable by the nitrate anion;
imbibing the aqueous solution onto a porous substrate, the porous substrate having a pore volume of at least 70%;
heating the imbibed porous substrate to a temperature sufficient to initiate an oxidation-reduction reaction between the nitrate anion and the phosphate-containing anion to form a solid and sufficient to incinerate the porous substrate; and
sintering the resulting solid at a temperature of at least 800° C. to form the bone graft material.

10. The method of claim 9 wherein the step of sintering the resulting solid occurs at a temperature of at least 1100° C. to form the bone graft material.

11. A bone graft material for the local delivery of strontium comprising strontium-doped calcium phosphate with from 0.1 to about 4.5 percent, by weight of the graft material, of strontium; the bone graft material having a pore volume of at least about 70%.

12. The bone graft material of claim 11 wherein the release of strontium ranges from about 3 ppm to about 145 ppm strontium per day for the first 12 weeks and from about 1 ppm to about 81 ppm strontium per day for the next 12 to 24 weeks post-implantation.

13. A method of inducing local bone formation in a defect site comprising the steps of:
accessing a defect site and
placing a strontium-doped calcium phosphate material within the defect site, wherein the strontium-doped calcium phosphate material includes from about 0.1 mol % to about 5 mol % strontium.
